(19) United States
(12) Patent Application Publication
(10) Pub. No.: US 2014/0163283 A1
(43) Pub. Date: Jun. 12, 2014

(54) METHOD OF CARRYING OUT CC-COUPLING REACTIONS USING OXIDE SUPPORTED PD-CATALYSTS

(71) Applicants: Eric Gaigneaux, Chastre (BE); Marc Jacquemin, Rochefort (BE); Damien Hauwaert, Nivelles (BE); Caroline Cellier, Chastre (BE); Alain Merschaert, Petitenghien (BE); Raquel Mateos Blanco, Zamora (ES)

(72) Inventors: Eric Gaigneaux, Chastre (BE); Marc Jacquemin, Rochefort (BE); Damien Hauwaert, Nivelles (BE); Caroline Cellier, Chastre (BE); Alain Merschaert, Petitenghien (BE); Raquel Mateos Blanco, Zamora (ES)

(73) Assignee: UNIVERSITE CATHOLIQUE DE LOUVAIN, Louvain La Neuve (BE)

(21) Appl. No.: 14/131,353

(22) PCT Filed: Jul. 6, 2012
(86) PCT No.: PCT/EP2012/063238

§ 371 Date: Jan. 7, 2014

(30) Foreign Application Priority Data
Jul. 7, 2011 (EP) 11173056.0

(51) Int. Cl. C07C 1/32

(52) U.S. Cl. CPC C07C 1/321
USPC 585/427

(57) ABSTRACT

A method of carrying out Suzuki-Miyaura CC-coupling reactions, includes reacting an aryl halide with an aryl boronic acid in an organic solvent in the presence of an oxide supported palladium catalyst and a base, characterized in that the base is added in the form of an aqueous solution with a constant flow rate within a predetermined period of time at the beginning of the reaction.

The present invention is directed to a method of carrying out C—C coupling reactions in the presence of a heterogeneous catalyst supported on oxide supports and a base.

### BACKGROUND OF THE INVENTION

Chemical reactions aiming to couple carbon atoms are important methodologies for the preparation of organic molecules. These reactions have recently emerged and are often a crucial step in the synthesis of many molecules, especially for the synthesis of pharmaceuticals such as Vancomycine (antibiotic), Steganone, Steganacine (anticancer) or Korupensamine (antimalarial). These reactions are also essential for the synthesis of materials such as liquid crystals, organic conductive materials and semi-conductors. The demand for these molecules is getting more and more important.

Suzuki-Miyaura's reaction was published at first in 1979 by 2010 Nobel Prize winner Akira Suzuki et al. (Miyaura N., Yamada K., Suzuki A., Tetrahedron Lett. 20, 1979, 3437). It creates an aryl-aryl bond in the presence of a palladium catalyst. It allows the combination of an aryl- or vinyl-boronic acid or esters thereof via the boronate group with vinyl or aryl halides (Felpin F.-X., Ayad T. and Mitra S., Eur. J. Org. Chem., 2006, 2679). The reactivity of the aryl halide depends very strongly on the nature of the halides: I>Br>>Cl. The iodides and bromides are usually used, on the other hand the chlorides are less reactive but more and more research is carried out about these molecules (Felpin F.-X., Ayad T. and Mitra S., Eur. J. Org. Chem., 2006, 2679; Simeone J. P., Sowa Jr. J. R., Tetrahedron 63, 2007, 12646-12654).

Suzuki-Miyaura's reaction is carried out in an organic solvent in the presence of a base, which is added at once the beginning of the reaction and consumed as the reaction goes on.

Suzuki-Miyaura's reaction is one of the most popular reactions for the production of biaryls for several reasons: (i) using mild conditions; (ii) using boron compounds which are stable, available and have a low toxicity and (iii) a wide range of substrates with various functional groups can be used (Felpin F.-X., Ayad T. and Mitra S., Eur. J. Org. Chem., 2006, 2679; Kotha S., Lahiri K., Kashinath D., Tetrahedron, 58, 2002, 9633).

Suzuki-Miyaura's reaction is traditionally carried out with homogeneous palladium catalysts. These catalysts are soluble complexes of palladium associated to ligands of the arylphosphine type such as the tetrakis(triphenylphosphine)palladium (Pd(PPh₃)₄) (Lu G., Franze R., Zhanga Q., Xua Y., Tetrahedron Lett. 46, 2005, 4255; Amatore, C., Pflüger, F. Organometallics 9, 1990, 2276). Good yields are obtained with these ligands but they often need a big quantity of catalysts (1 to 10 mol %).

The formation of carbon-carbon bonds under homogeneous catalysis has a high potential. Unfortunately, the use of soluble complexes of palladium shows important drawbacks. Firstly, it is very difficult to recover the homogeneous catalysts which are very expensive and it is also difficult to separate them from the reactants and products.

Therefore, this type of catalyst cannot be reused. Product contaminations by traces of dissolved catalyst remaining after separation can also arise. This point is particularly important in the synthesis of pharmaceutical molecules for which the residual metal tolerance is very low (less than 5 ppm). Gradually, organic chemistry is turning towards reusable heterogeneous catalysts for the economic and efficient use of raw materials. In addition to the abovementioned drawbacks, unwanted side reactions such as dehalogenation of aryl halide starting material are often encountered and working under inert atmosphere, if not mandatory, is preferable.

Heterogeneous catalysts have been developed to avoid the problems mentioned previously. The main advantages of those catalysts are that they can be recovered by simple filtration at the end of the reaction and that there is no more product contamination by metals. The catalyst can also be recycled for further reactions. The most common heterogeneous catalyst used for the Suzuki-Miyaura reaction is palladium supported on activated carbons (Pd/C) (Felpin F.-X., Ayad T. and Mitra S., Eur. J. Org. Chem., 2006, 2679; Simeone J. P., Sowa Jr. J. R., Tetrahedron 63, 2007, 12646-12654; Lu G., Franze R., Zhanga Q., Xua Y., Tetrahedron Lett. 46, 2005, 4255). However, the activity of those catalysts is very low.

The use of palladium supported on other materials than carbon, like silica, alumina, titanium oxide, and magnesium oxide can be found in the literature (Kabalka G. W., Wang L., Pagni R. M., Hair C. M., Namboodiri V., Synthesis, 2, 2003, 217; Budroni G., Corma A., García H., Primo A., J. Catal. 251, 2007, 345; A. Gniewek, J. J. Ziolkowsky, A. M. Trzeciak, M. Zawadzki, H. Grabowska, J. Wrzyszcz, J. Catal., 254, 2008, 121). The activity of those catalysts is however low and the conditions in which they are used are very harsh and dissolve the catalysts as the pH is too high.

As a summary, there is still a stringent need for improvements of the Suzuki-Miyaura reaction that combine the advantages of homogenous palladium catalysts and of supported palladium catalysts.

### SUMMARY OF THE INVENTION

After long and intensive research, the present inventors found that the activity of palladium on metal oxide catalysts in Suzuki-Miyaura reactions is significantly improved when the addition of the base needed for the reaction is controlled.

In a general aspect, the invention thus provides a method of carrying out CC-coupling reactions, preferably Suzuki-Miyaura reactions, comprising reacting an aryl halide with an aryl boronic acid, preferably phenylboronic acid, or an ester thereof, in an organic solvent in the presence of an oxide supported palladium catalyst and a base, characterized in that the base is added in the form of an aqueous solution with a constant flow rate within a predetermined period of time at the beginning of the reaction.

The supported catalyst used herein remains insoluble in the reaction medium, the method of the invention is thus in the field of heterogeneous catalytic reactions as opposed to homogeneous catalytic reactions wherein the catalyst is dissolved in the reaction medium.

### DETAILED DESCRIPTION OF THE INVENTION

As mentioned above, the invention relates to a method of carrying out CC-coupling reactions, preferably Suzuki-Miyaura reactions, comprising reacting an aryl halide with an aryl boronic acid or an ester thereof, in an organic solvent in the presence of an oxide supported palladium catalyst and a base, characterized in that the base is added in the form of an aqueous solution with a constant flow rate within a predetermined period of time at the beginning of the reaction.

More specifically, the Suzuki-Miyaura reactions according to the invention mean reacting a compound of general formula A with a compound of general formula B, in an organic solvent optionally in mixture with water, in the presence of an oxide supported palladium catalyst and a base, to furnish a compound of general formula C as depicted in scheme 1 below:

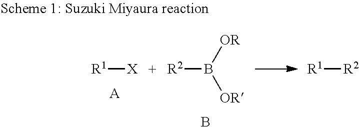

wherein R¹ is aryl;
X is halo;
R² is aryl;
R and R′, identical or different, are independently selected from H, linear or branched C1-C6 alkyl, or R and R′ form together a C2-C5 alkylene chain optionally substituted by one or more C1-C2 alkyl group, thus forming a cyclic boronate group, or R and R′ form together a phenylene ring wherein the oxygen atoms are attached at positions 1 and 2 thereof, thus forming a catechol boronate.

The term “halo” according to the invention means fluoro, chloro, bromo, or iodo. Preferred halo groups are bromo and iodo. More preferably, the halo group is bromo.

The term “halide” according to the invention means fluoride, chloride, bromide, or iodide. Preferred halide groups are bromide and iodide. More preferably, the halide is bromide.

The term “aryl” as used herein by itself or as part of another group refers to a substituted or unsubstituted polyunsaturated, aromatic hydrocarbyl group having a single ring (e.g. phenyl) or multiple aromatic rings fused together (e.g. naphtyl) or linked covalently, typically containing 5 to 12 atoms; preferably 6 to 10, wherein at least one ring is aromatic. Aryl is also intended to include the partially hydrogenated derivatives of the carbocyclic systems enumerated herein. Non-limiting examples of aryl comprise phenyl, tolyl, biphenylyl, biphenylenyl, 5- or 6-tetralinyl, naphthalen-1- or -2-yl, 4-, 5-, 6 or 7-indenyl, 1- 2-, 3-, 4- or 5-acenaphtylenyl, 3-, 4- or 5-acenaphtenyl, 1- or 2-pentalenyl, 4- or 5-indanyl, 5-, 6-, 7- or 8-tetrahydronaphthyl, 1,2,3,4-tetrahydronaphthyl, 1,4-dihydronaphthyl, 1-, 2-, 3-, 4- or 5-pyrenyl. Preferred aryl groups are phenyl, tolyl, and naphtyl, more preferably phenyl and tolyl.

As indicated above, the aryl moieties can be substituted by one or more substituent(s). Non limiting examples of substituents of aryl moieties are cyano, nitro, substituted and unsubstituted linear or branched C1-C6 alkyl, substituted and unsubstituted linear or branched C1-C6 alkoxy, hydroxyl, carboxaldehyde, carboxy, amino, amides, sulfonamides, ureas, carbamates, and derivatives thereof. Preferred substituents are C1-C2 alkyl, preferably methyl.

The expression “aryl halide” refers to an aryl radical having the meaning as defined above wherein one or more hydrogen are replaced with a halo as defined above. Non-limiting examples of such aryl halides include fluorobenzene, chlorobenzene, bromobenzene, iodobenzene, 2-bromotoluene, 3-bromotoluene, 4-bromotoluene, 2-iodotoluene, 3-iodotoluene, 4-iodotoluene.

The term “alkyl” by itself or as part of another substituent refers to a hydrocarbyl radical of Formula CₙH₂ₙ₊₁ wherein n is a number greater than or equal to 1. Generally, alkyl groups of this invention comprise from 1 to 6 carbon atoms, preferably from 1 to 4 carbon atoms, more preferably from 1 to 3 carbon atoms, still more preferably 1 to 2 carbon atoms. Alkyl groups may be linear or branched and may be substituted as indicated herein. Cx-Cy-alkyl refers to alkyl groups which comprise from x to y carbon atoms.

When the suffix “ene” (“alkylene”) is used in conjunction with an alkyl group, this is intended to mean the alkyl group as defined herein having two single bonds as points of attachment to other groups. The term “alkylene” includes ethylene, propylene, and pentylene.

The term “boronic acid” refers to a compound bearing a

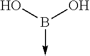

group, wherein the arrow designates the attachment point.

The expression “aryl boronic acid” alone or in combination refers to an aryl radical having the meaning as defined above wherein one or more hydrogen are replaced with a boronic acid as defined above. An examples of such aryl boronic acids is phenyl boronic acid.

The expression “ester of boronic acid” refers to a compound bearing a

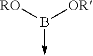

group, wherein the arrow designates the attachment point, and wherein R and R′, are defined as in Formula B above.

Suitable esters of boronic acids are di-(linear or branched C1-C4 alkyl)boronate derivatives, for instance dimethoxyboronate or dicthoxyboronates, and boronate esters of 1,3-propanediol, 1,1,2,2-tetramethylethan-1,2-diol (pinacol), 2-methyl-2,4-pentanediol and catechol (2-hydroxyphenol), the boronate ester of pinacol being the preferred ester of boronic acid.

Traditionally, the base is added in solid form at once in an uncontrolled manner at the beginning of the reaction and then consumed as the reaction goes on. The expression “in an uncontrolled manner” as used herein means that the conditions under which the base is added may vary from one batch to the other in terms of dissolution or flow rate and duration of addition.

Adding the base in the form of an aqueous solution at a constant flow rate within a predetermined period of time at the beginning of the reaction under heterogenous catalysis improves the conversion rate and the selectivity of the Suzuki-Miyaura reaction compared to adding the base in an uncontrolled manner. The selectivity is defined as the ratio between the number of moles of 4-methylbiphenyl produced and the number of moles of 4-bromotoluene converted. The invention thus provides a method of carrying out Suzuki-Miyaura reactions that combines the advantages of heterogeous catalysis with those of homogenous catalysis: easy recovery (e.g. by filtration) and recycling of the catalyst due to the use of a supported catalyst and increased activity of the catalysts leading to higher selectivity and conversion rates compared to traditional heterogenous catalysis.

Furthermore, the controlled addition of the base within a predetermined period of time is absolutely detrimental for the efficacy of the process of the invention. When the base is added at once, at the same time as the other reagents, the catalyst coagulates and gets deposited onto the side walls of the reaction vessel. Its catalytic activity is thus annihilated and the reaction does not even process at all.

According to an advantageous embodiment of the invention, the aqueous solution of the base has a concentration of 0.04 mol/L to 1.1 mol/L, preferably 0.14 mol/L to 0.8 mol/L and even more preferably of about 0.4 mol/L. Preferably, the solution of the base is buffered at a pH of 8 to 12, preferably 10 to 11, more preferably about 10.6. Any suitable acid can be used for buffering the basic solution, such as HCl, H₂SO₄, HBr and HCl being preferred.

The base is advantageously added at a flow rate of 0.133 to 0.2 L/(min*L reactor volume), preferably 0.150 to 0.183 L/(min*L reactor volume) and even more preferably about 0.167 L/(min*L reactor volume).

The base is advantageously added within a period of time of 1 to 2 minutes, preferably 1.25 to 1.55 minutes and even more preferably about 1.5 minutes.

The base is preferably an inorganic base. Suitable inorganic bases include, but are not limited to K₂CO₃, Na₂CO₃, Cs₂CO₃, NaOH, KOH, Na₃PO₄, and KF. In a preferred embodiment, the base is selected from the group consisting of K₂CO₃, NaOH, KOH, and Na₂CO₃.

A wide variety of organic solvents can be used for Suzuki-Miyaura reactions. Suitable organic solvents include, but are not limited to, DMF (dimethylformamide), DME (dimethoxyethane), DMA (dimethylacetamide), NMP (N-methylpyrrolidone), THF (tetrahydrofuran), toluene, methanol, ethanol, iso-propanol, n-butanol, water, and mixtures thereof. In a preferred embodiment, the solvent is DMF, alone or in a mixture with water. More preferably, the solvent is a mixture of DMF and water.

Suitable oxide supports include those presenting acido-basic, redox, amphotcrc and/or hydrophilic/hydrophobic properties, preferentially acido-basic properties. Oxide supports include but are not limited to γ-Al₂O₃, TiO₂, ZrO₂, CeO₂, MgO, SiO₂, and SiO₂—P. Preferably the oxide support is TiO₂.

Advantageously, the palladium is loaded on the oxide support in a quantity of 0.3 to 10%, preferably 3 to 6% and even more preferably about 5% by weight with respect to the weight of the support.

The CC-coupling reaction is advantageously carried out at a temperature of between 70° C. and 100° C., preferably between 78° C. and 98° C., even more preferably between 80° C. and 95° C. and most preferably about 95° C.

Excellent results are obtained with a Pd on TiO₂ catalyst and an aqueous solution of K₂CO₃ having a concentration of 0.05 g/mL and which was buffered at a pH of about 10.6. The reaction is advantageously carried out at a temperature of about 95° C.

The catalysts used in the method of the invention can be prepared according to the methods known in the art, such as wet impregnation, deposition-precipitation, grafting or a mix of wet impregnation and grafting.

Owing to the oxide support catalysts which are not sensitive to air oxidation and wherein the active Pd is at a +2 oxidation state, the method of the invention does not require to work under inert conditions. This method is thus very versatile when compared to traditional homogeneous catalysts methodologies which often require to work under a nitrogen or argon atmosphere.

Furthermore, as shown in the examples, no dehalogenation of the aryl halide starting material was observed.

The present invention will be better understood with reference to the following examples. These examples are intended to be representative of specific embodiments of the invention, and are not intended as limiting the scope of the invention.

### EXAMPLES

### Example 1

### Preparation of the Catalysts

The following supports were used:
γ-Al₂O₃ (Alfa Aesar, 039812),
TiO₂ (Degussa, P25),
ZrO₂ (Acros, 19052-2500),
CeO₂ (Janssen Chemica, 1991227),
MgO (D. Chen, E. H. Jordan, Mater. Lett., 63, 2009, 783),
SiO₂ (R. E. Sempels and P. G. Rouxhet, J. Colloid Interface Sci., vol 55, No. 2, 1976),
SiO₂-A (Sigma-Aldrich, 236845),
SiO₂—P which is a silica called “Perlite” and modified by alkalis and alkaline earth elements, like alumina, titania, sodium (Evonik, Sipernat 22).
   Before synthesis, the supports were calcined in air for 15 hours at 500° C. The quantity of the precursor used was adapted to obtain 5% in weight of Pd compared to the oxide support.

### 1.1. Wet Impregnation (WI)

Palladium tetraamine chloride (Pd(NH₃)₄Cl₂.H₂O, Aldrich, 323438) was dissolved in distilled water and the pH was adjusted with ammonia (33%) until 10.6. 4 g of calcined support was mixed with the solution for 1 hour under magnetic stirring. Water was then evaporated under reduced pressure in a rotavapor at 40° C. The recovered solid was dried in air for one night at 110° C. and calcined at 500° C. for 3 hours in a muffle furnace under static air.

### 1.2. Deposition-Precipitation (DP)

For the synthesis of those catalysts, the salt of palladium used is palladium nitrate (Pd(NO₃)₂, Aldrich, 205761). The precursor was dissolved in distilled water to obtain a solution having a concentration of 14 mmol/L of Pd (solution A). A second aqueous solution of NaOH (140 mmol/L) (solution B) was prepared. 4 g of the calcined support, 20 mL of distilled water and 4 mL of solution A were mixed and the solution B was added drop by drop. All is mixed during 1 hour. The recovered solid was centrifuged and washed three times with distilled water and then dried for one night at 110° C. and calcined at 500° C. for 3 hours in a muffle furnace under static air.

### 1.3. Grafting (G)

The catalysts prepared by grafting were only prepared with silica from Sigma-Aldrich (236845). 2 g of calcined support was added to a solution in which the palladium bis-acetylacetonate (Pd(C₅H₇O₂)₂, Aldrich, 209015) was dissolved in 10 mL of toluene. Two temperatures for the synthesis have been tried: room temperature or reflux (>110.6° C.). Three stirring durations have been tested: 4, 8 and 16 h. The recovered solid was washed three times with distilled waterunder Büchner and then dried for 18 hours at 70° C. or 110° C.

### 1.4. Mix of Wet Impregnation and Grafting (WIG)

After 18 h of stirring, the solvent was totally evaporated and the recovered solid was washed with 220 mL of distilled water under Büchner and then dried for 18 hours at 110° C. and calcined at 500° C. for 3 hours in a muffle furnace under static air.

Catalysts are denoted Pd(5%)/ab where “a” represents the support and “b” is the method used during the preparation (WI=wet impregnation, DP=deposition-precipitation, WIG=mix of wet impregnation and grafting and (G=grafting).

The following catalysts were prepared:

### γ-Al₂O₃

Pd(5%)/Al₂O₃ WI
Pd(5%)/Al₂O₃ DP
Pd(5%)/Al₂O₃ WIG

### TiO₂

Pd(5%)/TiO₂ WI
Pd(5%)/TiO₂ DP
Pd(5%)/TiO₂ WIG

### SiO₂

Pd(5%)/SiO₂ WI
Pd(5%)/SiO₂ DP
Pd(5%)/SiO₂ WIG

### SiO₂-A

Pd(5%)/SiO₂-A G (samples 1 to 12)

### SiO₂—P

Pd(5%)/SiO₂—P WI
Pd(5%)/SiO₂—P DP
Pd(5%)/SiO₂—P WIG

### ZrO₂

Pd(5%)/ZrO₂ WI
Pd(5%)/ZrO₂ DP
Pd(5%)/ZrO₂ WIG

### MgO

Pd(5%)/MgO WI
Pd(5%)/MgO DP
Pd(5%)/MgO WIG

### CeO₂

Pd(5%)/CeO₂ WI
Pd(5%)/CeO₂ DP
Pd(5%)/CeO₂ WIG

### Example 2

### Characterization of Catalysts

The chemical composition of catalysts was measured by Inductively Coupled Plasma Atomic Emission Spectroscopy (ICP-AES) on an Iris Advantage apparatus from Jarrel Ash Corporation. The catalyst was dried at 110° C. prior to measurement.

Textural analysis of the catalyst was carried out on a Micromeritics Tristar 3000 equipment using N₂ adsorption/desorption at liquid N₂ temperature, working with relative P/P₀ pressures in the range of 10⁻² to 1.0. Before the measurements, 150 mg of the samples were degassed at 150° C. overnight under a vacuum (50 mTorr). The specific surface area was calculated from the amount of N₂ adsorbed by using 5 points with relative P/Pₒ pressures between 5*10⁻² and 0.3 (BET theory). BJH equations were used to determine the distribution of pores diameter and the total pore volume was assessed from the amount of nitrogen adsorbed at P/P₀=0.98.

X-ray diffraction (XRD) analysis were performed on the fresh catalyst on a Siemens D5000 diffractometer using the K_{α} radiation of Cu (λ=1.5418 Å). The 2θ range was scanned between 5 and 90° at a rate of 0.02°.s⁻¹. Identification of the crystalline phases was carried out using the ICDD-JCPDS database.

Surface characterization of the fresh catalyst was done by X-ray photoelectron spectroscopy (XPS) measurements on a Kratos Axis Ultra spectrometer (Kratos Analytical—Manchester—UK) equipped with a monochromatized aluminium X-ray source (powered at 10 mA and 15 KV).

Dispersion of Pd was determined by using carbon monoxide chemisorption. The CO chemisorption measurements were conducted at 35° C. using a Micromeritics Pulses Chemisorb 2700 apparatus equipped with a TCD detector. The sample (150 mg) was reduced at 400° C. under a pure flow of Hydrogen (Praxair, 99.999%) for 2 hours and then flushed for 1 hour under He and finally cooled down to 35° C. Several injections of a known volume of CO (185 μl) are sent on the samples. The apparatus gives a peak area which corresponds to the non-adsorbed CO. When there is no more adsorption of CO, the value of the peak area corresponds to the volume of the injection loop. By simple subtraction, the volume of CO which was adsorbed at each injection on palladium was calculated. Thereafter, those values were added for all injections of CO to obtain the total volume of CO adsorbed. This total is corrected so as to adjust the standard conditions of pressure and temperature. The stoechiometry of adsorption is equaled to one adsorbed CO molecule per atom of active metal (Pd).

The quantity of CO chemisorbed is related to the dispersion (D) with the following equation:

$$D = \frac{\sum Δ S \cdot {V}_{co} \cdot MM \cdot A}{{S}_{max} \cdot B \cdot m}$$

### With:

ΣΔS: the sum of the difference between the values of area measured before the saturation of the sample and the value of the maximal area which corresponds to the saturation,
V_{CO}: the volume injected CO (1),
A: a conversion factor (273/(298×22.4)) (mol·l⁻¹),
MM: molar mass of the metal (106.42 g·mol⁻¹),
S_{MAX}: area of the peak which corresponds to the volume of CO injected in one injection,
B: loading of the metal deposed on the support (%),
m: mass of the analyzed sample (g).

The particle size of the Pd on the catalysts is estimated by the following equation:

$$d ( m ) = \frac{500 \times MM}{ρ \times σ \times D}$$

d: Particle size (m)
MM: Molar mass of the metal (Pd: 106.42 g·mol⁻¹);
ρ: Density of the metal (Pd: 12×10⁶ g·m⁻³);
σ: Surface of one mole of the metal (Pd: 47800 m²ₘₑₜₐₗ.mol⁻¹);
D: Dispersion (%).

The results of chemical analyses performed on oxides supported catalysts are presented in table 1. Dispersions (%) and the Pd particles size (nm) are also presented.

TABLE 1
Chemical composition on catalysts supported oxides supports
Prepared by WI, DP and WIG

| | Pd (wt. %) | Dispersion | Particles size |
|---|---|---|---|
| Catalysts | experimental* | (%) | (nm) |
| Pd(5%)/Al₂O₃ WI | 5.8 | 7 | 13 |
| Pd(5%)/Al₂O₃ DP | 0.2 | 7 | 13 |
| Pd(5%)/Al₂O₃ WIG | 3.4 | 12 | 8 |
| Pd(5%)/TiO₂ WI | 5.2 | 1 | 93 |
| Pd(5%)/TiO₂ DP | 4.8 | 11 | 8 |
| Pd(5%)/TiO₂ WIG | 4.0 | 5 | 19 |
| Pd(5%)/SiO₂ WI | 4.1 | 12 | 8 |
| Pd(5%)/SiO₂ DP | 0.2 | 19 | 5 |
| Pd(5%)/SiO₂ WIG | 4.2 | 19 | 5 |
| Pd(5%)/SiO₂-P WI | 4.3 | 7 | 13 |
| Pd(5%)/SiO₂-P DP | 3.6 | 2 | 46 |
| Pd(5%)/SiO₂-P WIG | 4.3 | 15 | 6 |
| Pd(5%)/ZrO₂ WI | 5.2 | 7 | 13 |
| Pd(5%)/ZrO₂ DP | 2.3 | 11 | 8 |
| Pd(5%)/ZrO₂ WIG | 4.4 | 10 | 9 |
| Pd(5%)/MgO WI | 3.1 | 9 | 10 |
| Pd(5%)/MgO DP | 4.1 | 9 | 10 |
| Pd(5%)/MgO WIG | 3.8 | 16 | 6 |
| Pd(5%)/CeO₂ WI | 5.7 | 3 | 31 |
| Pd(5%)/CeO₂ DP | 1.5 | 3 | 31 |
| Pd(5%)/CeO₂ WIG | 4.2 | 3 | 31 |

*in mass compared to the support

The amount of Pd on the catalysts prepared by deposition-precipitation is very low. Indeed, the experimental measurements are quite different from the theoretical loading. The palladium is probably lost during the centrifugation steps during the synthesis. Concerning the WIG, the experimental loading is about 4%. The role of the support is very important, the dispersion changes according to its nature. In wet impregnation, the dispersion remains more or less uniform while for the WIG and the deposition-precipitation, the dispersion could be very different according to the nature of the support and the synthesis method. This confirms that the deposition-precipitation and the WIG involve the surface groups more than the WI.

Table 2 shows that the specific surface area (SSA) for the starting supports and the catalysts on oxide supports.

TABLE 2
Textural analysis (BET) of the oxides supports and catalysts
supported on oxides supports prepared by WI, DP, WIG

| Supports and | Specific surface | Porous volume | Pores size |
|---|---|---|---|
| Catalysts | area (m²/g) | (cm³/g) | (\acute{Å}) |
| Al₂O₃ | 72 | 0.22 | 122 |
| Pd(5%)/Al₂O₃ WI | 60 | 0.21 | 136 |
| Pd(5%)/Al₂O₃ DP | 59 | 0.29 | 200 |
| Pd(5%)/Al₂O₃ WIG | 70 | 0.28 | 161 |
| TiO₂ | 52 | 0.18 | 136 |
| Pd(5%)/TiO₂ WI | 56 | 0.32 | 227 |
| Pd(5%)/TiO₂ DP | 50 | 0.29 | 204 |
| Pd(5%)/TiO₂ WIG | 45 | 0.30 | 220 |
| SiO₂ | 481 | 1.03 | 69 |
| Pd(5%)/SiO₂ WI | 377 | 0.90 | 76 |
| Pd(5%)/SiO₂ DP | 430 | 0.96 | 69 |
| Pd(5%)/SiO₂ WIG | 436 | 1.00 | 74 |
| SiO₂-P | 171 | 1.10 | 276 |
| Pd(5%)/SiO₂-P WI | 165 | 1.04 | 294 |
| Pd(5%)/SiO₂-P DP | 177 | 1.03 | 210 |
| Pd(5%)/SiO₂-P WIG | 170 | 0.92 | 249 |
| ZrO₂ | 68 | 0.75 | 369 |
| Pd(5%)/ZrO₂ WI | 34 | 0.21 | 201 |
| Pd(5%)/ZrO₂ DP | 46 | 0.41 | 274 |
| Pd(5%)/ZrO₂ WIG | 51 | 0.22 | 138 |
| MgO | 84 | 0.35 | 127 |
| Pd(5%)/MgO WI | 36 | 0.13 | 171 |
| Pd(5%)/MgO DP | 59 | 0.47 | 337 |
| Pd(5%)/MgO WIG | 150 | 0.58 | 99 |
| CeO₂ | 3 | 0.01 | 128 |
| Pd(5%)/CeO₂ WI | 4 | 0.02 | 209 |
| Pd(5%)/CeO₂ DP | 5 | 0.01 | 138 |
| Pd(5%)/CeO₂ WIG | 4 | 0.02 | 164 |

All samples were characterized by XRD analysis. The crystalline phases detected are presented in table 3.

TABLE 3
XRD experiments on the catalysts supported on oxides supports

| | Crystalline of the | Crystalline phase of |
|---|---|---|
| Catalysts | support | Pd |
| Al₂O₃ | γ-Alumina | — |
| Pd(5%)/Al₂O₃ WI | γ-Alumina | PdO palladinite |
| Pd(5%)/Al₂O₃ DP | γ-Alumina | Amorphous |
| Pd(5%)/Al₂O₃ WIG | γ-Alumina | PdO palladinite |
| TiO₂ | Anatase + Rutile | — |
| Pd(5%)/TiO₂ WI | Anatase + Rutile | PdO palladinite |
| Pd(5%)/TiO₂ DP | Anatase + Rutile | Amorphous |
| Pd(5%)/TiO₂ WIG | Anatase + Rutile | PdO palladinite |
| SiO₂ | Amorphous | — |
| Pd(5%)/SiO₂ WI | Amorphous | PdO palladinite |
| Pd(5%)/SiO₂ DP | Amorphous | Amorphous |
| Pd(5%)/SiO₂ WIG | Amorphous | PdO palladinite |
| SiO₂ p | Amorphous | — |
| Pd(5%)/SiO₂ p WI | Amorphous | PdO palladinite |
| Pd(5%)/SiO₂ p DP | Amorphous | Amorphous |
| Pd(5%)/SiO₂ p WIG | Amorphous | PdO palladinite |
| ZrO₂ | ZrO₂ | — |
| Pd(5%)/ZrO₂ WI | ZrO₂ | PdO palladinite |
| Pd(5%)/ZrO₂ DP | ZrO₂ | Amorphous |
| Pd(5%)/ZrO₂ WIG | ZrO₂ | PdO palladinite |
| MgO | Periclase | — |
| Pd(5%)//MgO WI | Periclase | PdO palladinite |
| Pd(5%)/MgO DP | Periclase | Amorphous |
| Pd(5%)/MgO WIG | Periclase | PdO palladinite |
| CeO₂ | Cerine | — |
| Pd(5%)/CeO₂ WI | Cerine | PdO palladinite |
| Pd(5%)/CeO₂ DP | Cerine | Amorphous |
| Pd(5%)/CeO₂ WIG | Cerine | PdO palladinite |

The diffractograms show that the Pd is in the form of PdO on all the catalysts prepared by wet impregnation and WIG while for the deposition-precipitation, no peak corresponding to Pd was detected. This fact does not suggest that the Pd is not in crystalline form. It is possible that crystalline domains are present but undetected because they are too small.

TABLE 4
Chemical composition on catalysts supported
on SiO₂-A prepared by grafting

| | Pd (wt. %) | Dispersion | Particles size |
|---|---|---|---|
| Catalysts* | experimental** | (%) | (nm) |
| Pd(5%)/SiO₂-A G 1 | 1.1 | 14 | 7 |
| Pd(5%)/SiO₂-A G 2 | 1.1 | 13 | 7 |
| Pd(5%)/SiO₂-A G 3 | 0.3 | 25 | 4 |
| Pd(5%)/SiO₂-A G 4 | 0.3 | 22 | 4 |
| Pd(5%)/SiO₂-A G 5 | 0.8 | 14 | 7 |
| Pd(5%)/SiO₂-A G 6 | 0.8 | 10 | 9 |
| Pd(5%)/SiO₂-A G 7 | 0.8 | 4 | 21 |
| Pd(5%)/SiO₂-A G 8 | 0.8 | 11 | 9 |
| Pd(5%)/SiO₂-A G 9 | 0.9 | 12 | 8 |
| Pd(5%)/SiO₂-A G 10 | 0.9 | 9 | 10 |
| Pd(5%)/SiO₂-A G 11 | 0.7 | 5 | 18 |
| Pd(5%)/SiO₂-A G 12 | 0.7 | 9 | 10 |

*samples 1, 3, 5, 7, 9, 11 were dried at 70° C. and samples 2, 4,6, 8, 10, 12 at 110° C.
**In mass compared to the support

The amount of Pd on the catalysts prepared by grafting is very low. Indeed, the experimental measurements are quite different from the theoretical loading. The palladium is probably not grafted and lost during the filtration steps during the synthesis. However, the dispersion is generally high which means that the Pd particles have a small particle size.

Table 5 shows that the specific surface area (SSA) for the starting supports and the catalysts on oxide supports prepared by grafting.

TABLE 5
Textural analysis (BET) of the oxides supports and catalysts
supported on oxides supports prepared by grafting

| | Specific surface | Porous volume | Pores size |
|---|---|---|---|
| Supports and Catalysts* | area (m²/g) | (cm³/g) | (\acute{Å}) |
| SiO₂-A | 280 | 1.56 | 160 |
| Pd(5%)/SiO₂-A G 1 | 302 | 1.04 | 111 |
| Pd(5%)/SiO₂-A G 2 | 284 | 1.21 | 138 |
| Pd(5%)/SiO₂-A G 3 | 325 | 1.14 | 111 |
| Pd(5%)/SiO₂-A G 4 | 318 | 1.12 | 112 |
| Pd(5%)/SiO₂-A G 5 | 308 | 1.11 | 114 |
| Pd(5%)/SiO₂-A G 6 | 306 | 1.06 | 111 |
| Pd(5%)/SiO₂-A G 7 | 303 | 1.05 | 110 |
| Pd(5%)/SiO₂-A G 8 | 278 | 1.09 | 119 |
| Pd(5%)/SiO₂-A G 9 | 310 | 1.03 | 106 |
| Pd(5%)/SiO₂-A G 10 | 316 | 1.04 | 106 |
| Pd(5%)/SiO₂-A G 11 | 306 | 1.05 | 112 |
| Pd(5%)/SiO₂-A G 12 | 305 | 1.10 | 113 |

*samples 1, 3, 5, 7, 9, 11 were dried at 70° C. and samples 2, 4, 6, 8, 10, 12 at 110° C.

No peak corresponding to Pd was detected in XRD measurements. This fact does not suggest that the Pd is not in crystalline form. It is possible that crystalline domains are present but undetected because they are too small.

### Example 3

### Catalytic Tests

Suzuki-Miyaura's Catalytic Tests Reactions

The twenty one Pd/oxide catalysts prepared in Example 1 were tested in the following Suzuki-Miyaura test reaction (coupling reaction 1). The reactants were 4-bromotoluene and phenylboronic acid. The desired product is 4-methylbiphenyl.

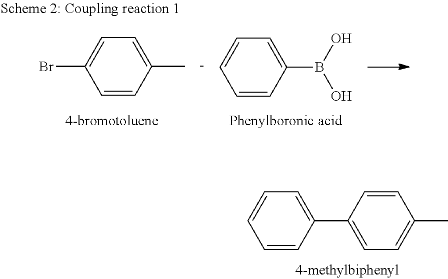

A 5 necked round bottom flask was placed in an oil bath. To reduce the loss of reagents by evaporation, a condenser is connected to the reactor. The reaction temperature was measured using a thermometer in contact with the reaction medium.

All catalysts were sieved and selected in the 100-200 μm granulometric fractions. The catalytic tests were carried out in the 5 necked flask under mechanical stirring (210 rpm). The solid reagents, 1.5000 g of 4-bromotoluene and 1.6041 g of phenylboronic acid, 0.1750 g of catalyst and 1.0810 g of biphenyl, the internal standard, were introduced first, followed by 60 mL of dimethylformamide (DMF). The flask was placed in a thermostatic oil bath and the reaction was performed under a nitrogen atmosphere. All catalytic tests have been realized by using a solution of K₂CO₃ (5 g of K₂CO₃/100 ml of water buffered with HCl at 10.6) and a reaction temperature of 95° C.

Once the working temperature (95° C.) was reached, 15 mL of the K₂CO₃ solution was added using a peristaltic pump within 1.5±0.10 min.

The reaction begins when the K₂CO₃ solution was added. Samples (0.5 mL) were taken every 1 min 30 sec. after adding the base until 10 min30 and after a last aliquot was taken at 15 min. Before analysis, samples are filtered (PTFE syringe filters, filtration threshold 0.45 μm) to remove the catalyst.

For quantification, an internal standard (biphenyl) was used and an appropriate calibration has been performed. Biphenyl was chosen as internal standard because it has a chemical structure very close to the desired reaction product, namely 4-methylbiphenyl. Tests were conducted to check that the internal standard did not react with any compounds present in the reaction, namely 4-bromotoluene, phenylboronic acid, 4-methylbiphenyl, the base and DMF. For the analysis of samples, the sample was diluted (1:9 vol.) in dichloromethane (JANSSEN CHIMICA, 1134696) is conducted and analyzed by a CP-3800 gas chromatography (GC) apparatus from Varian equipped with an autosampler (CP-8200 Varian) on a column CP-Sil 5CB (50 m×0.32 mm×0.4 μm) Varian. The initial pressure is 15 psi in the column. The temperature program applied for the separation of reactants and products in the chromatography column is as follows: initial temperature 80° C. is maintained 3 min, and then a ramp of 30° C./min was applied up to 180° C. This temperature is maintained for 3 minutes. A ramp of 15° C./min is applied to 250° C. for 3 min Finally, the temperature increases to 300° C. with a ramp of 30° C./min. This temperature is maintained during 3 min. The total duration of the run is 15.76 minutes. The conversion is defined as the ratio “moles of 4-bromotoluene converted/mole of 4-bromotoluene initial*100%”. The selectivity for the 4-methylbiphenyl is defined as the ratio “moles of 4-methylbiphenyl produced/mole of 4-bromotoluene consumed*100%”.

### Test Results

a. Performance of the catalysts

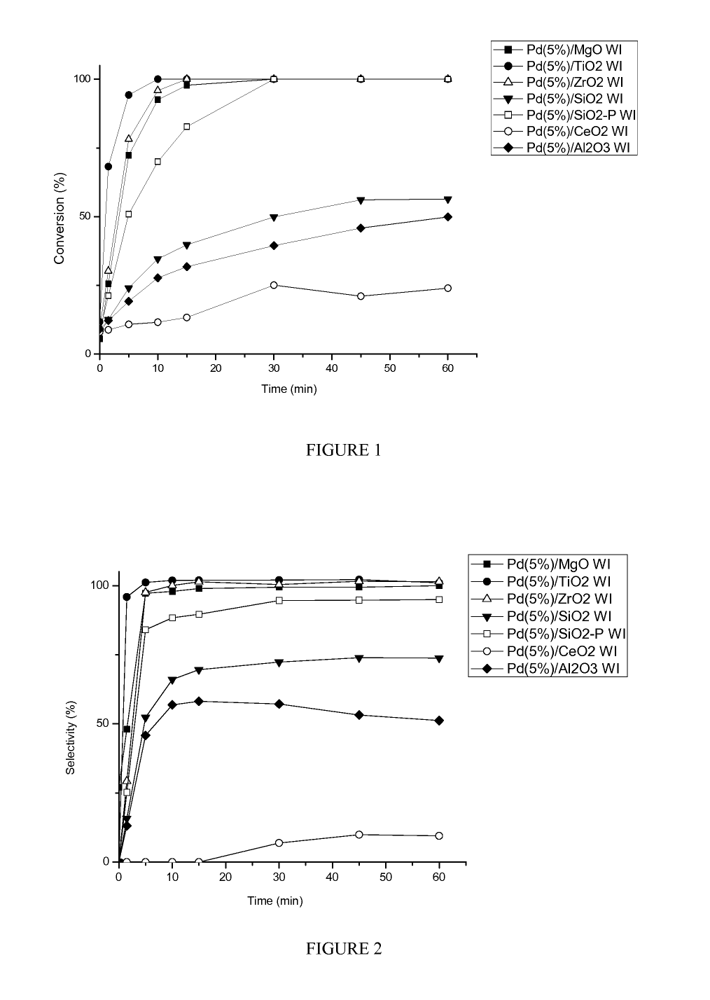

FIG. 1 and FIG. 2 show the performances obtained with the catalysts prepared by wet impregnation.

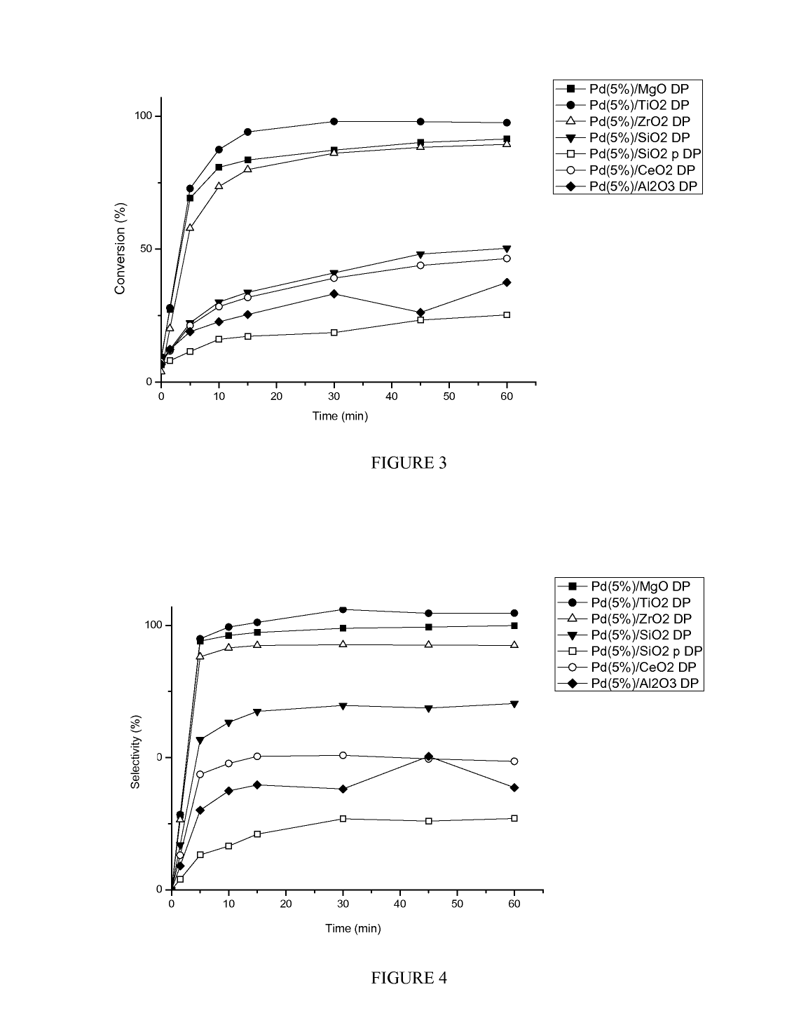

FIG. 3 and FIG. 4 show the performances obtained with the catalysts prepared by deposition-precipitation.

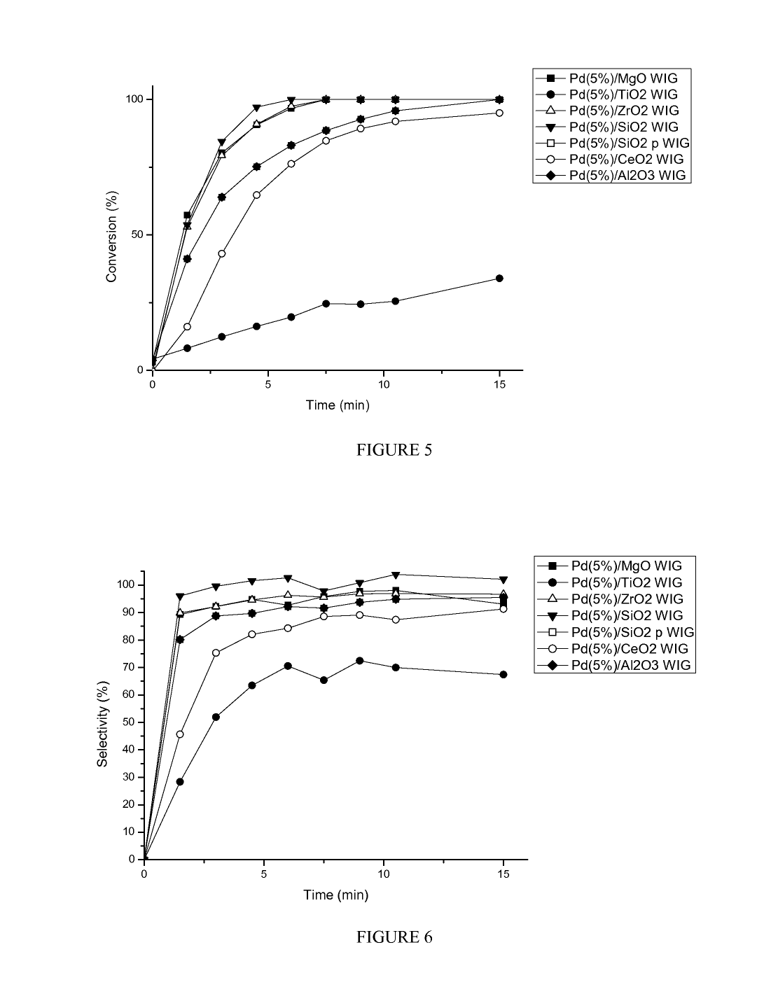

FIG. 5 and FIG. 6 show the performances obtained with the catalysts prepared by WIG.

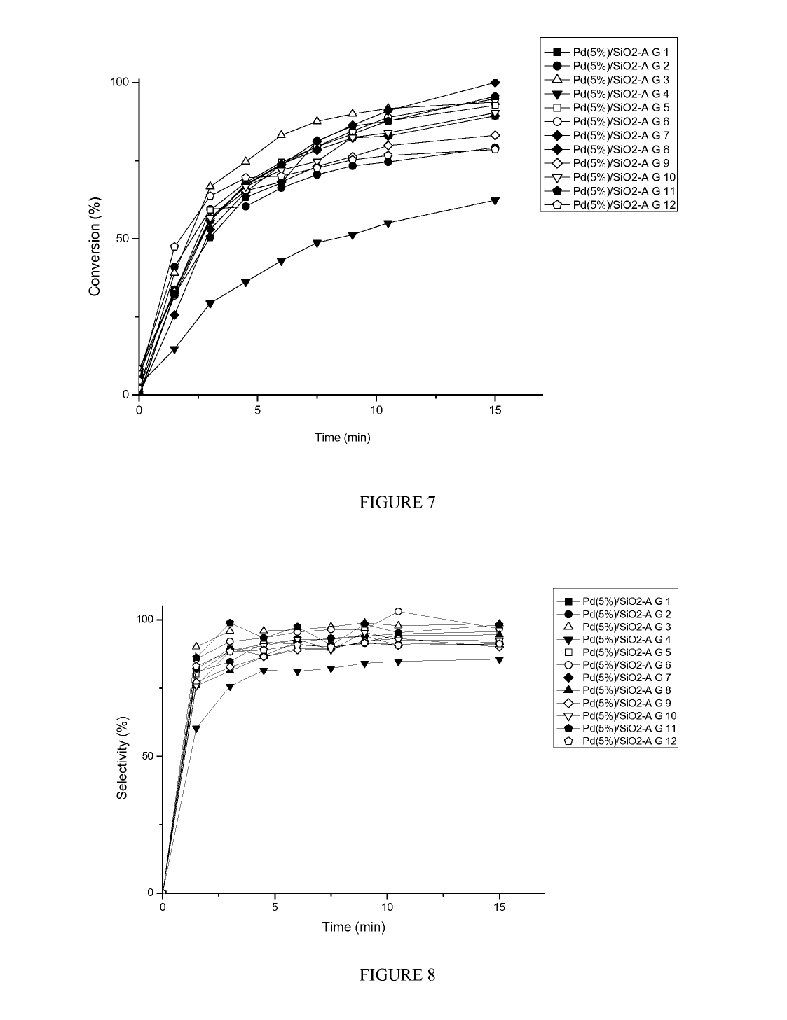

FIG. 7 and FIG. 8 show the performances obtained with the catalysts prepared by grafting.

All the catalysts prepared by wet impregnation, deposition-precipitation, WIG and grafting have very good performances (FIGS. 1 to 8). The reaction is very fast with very good conversion and selectivity.

Pd/TiO₂ 5% WI showed the best catalytic performance.

b. Influence of the Temperature Reaction on the Activity

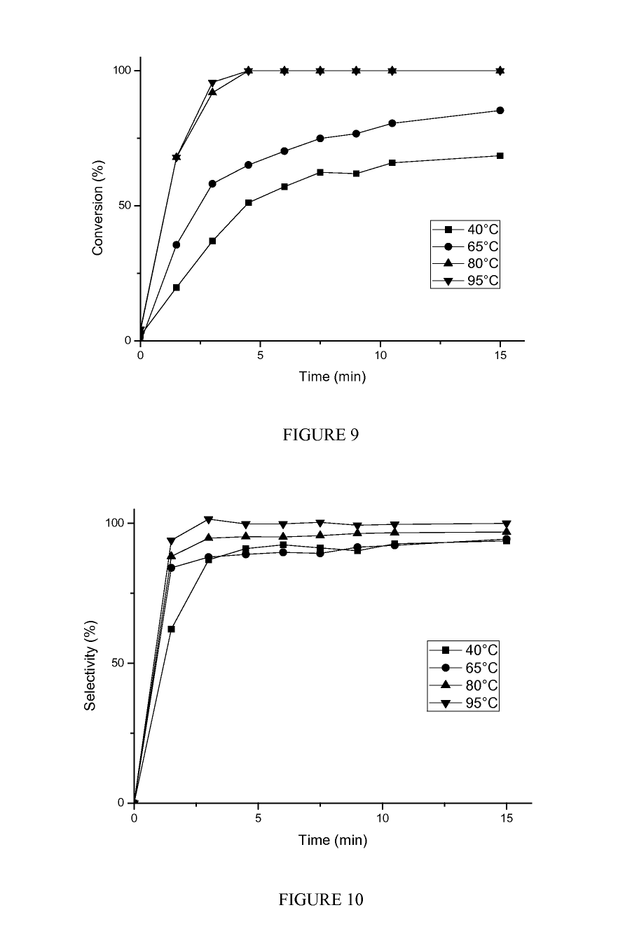

Different temperatures of reaction have been tested (40, 65, 85 and 95° C.) using the procedure described above. The best performance including the selectivity to 4-methylbiphenyl was obtained when the temperature was about 95° C. (FIGS. 9 and 10).

c. Influence of the Nature of the Base on the Activity:

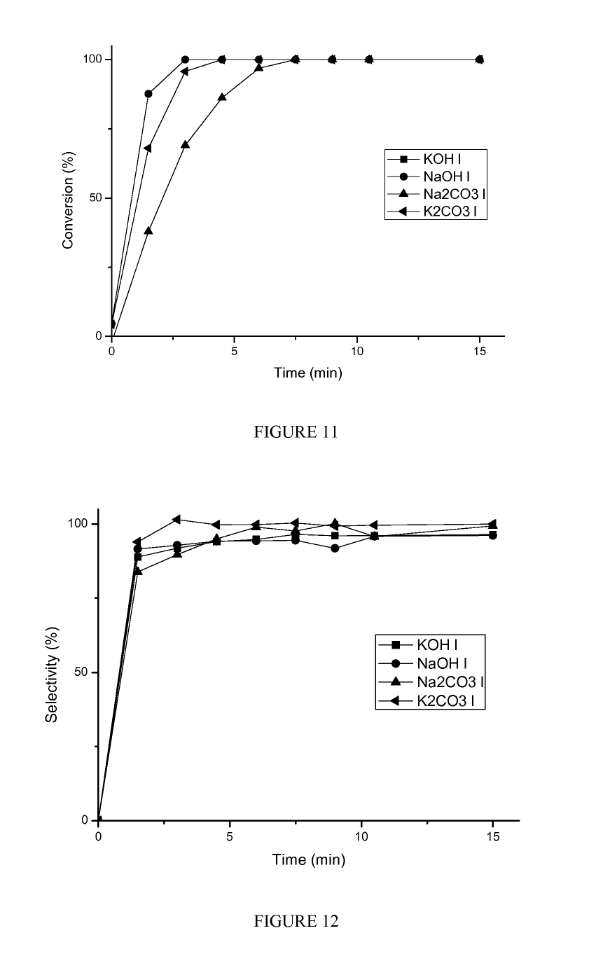

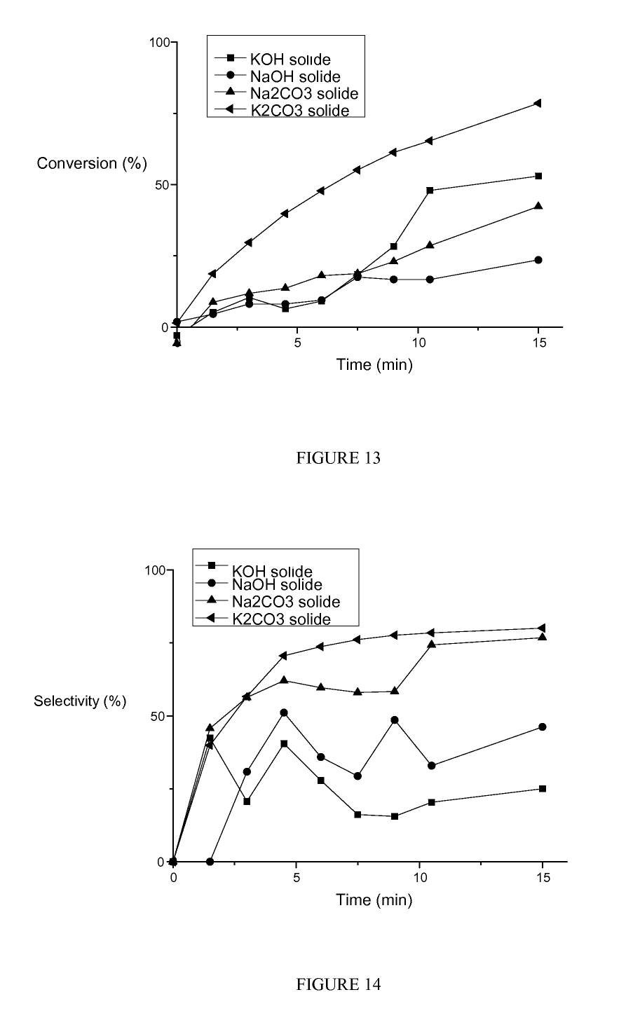

Different kinds of base have been tested (K₂CO₃, NaOH, KOH, Na₂CO₃) using the procedure described above. These salts were dissolved in water and the pH of the solution was adjusted until 10.6 with HCl (FIGS. 11 and 12) or the salts were added directly in the reaction (FIGS. 13 and 14). The performances are generally better when using an aqueous solution of the base and the best performance was obtained for K₂CO₃ diluted in water.

Hence, adding an aqueous solution of a base, especially K₂CO₃ at a constant flow rate within a predetermined time improves the selectivity of Suzuki-Miyaura reactions while maintaining equivalent conversion rates.

d. Influence of the Base Addition

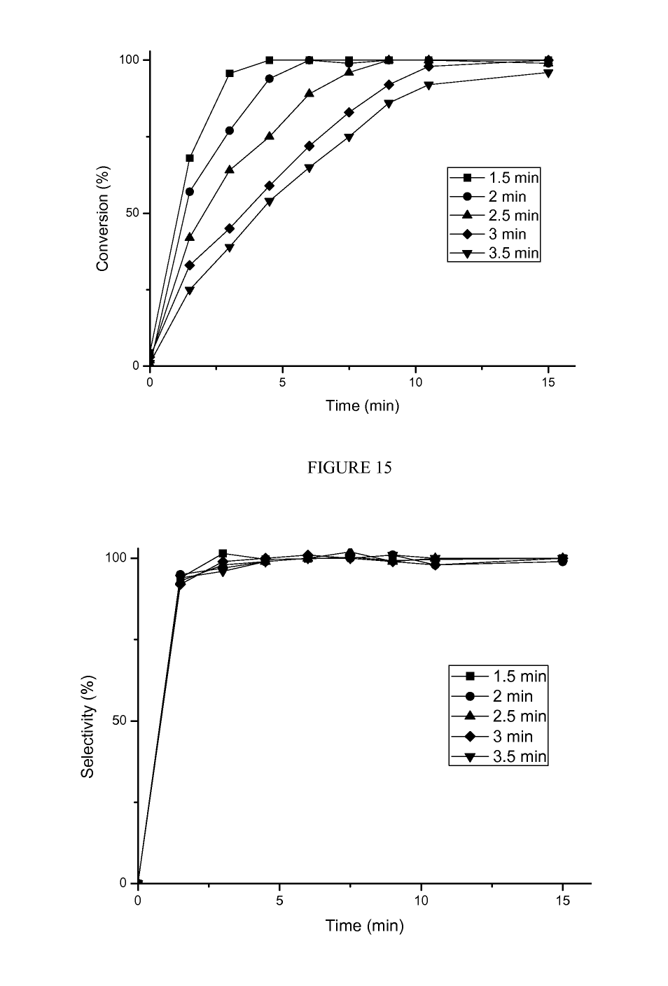

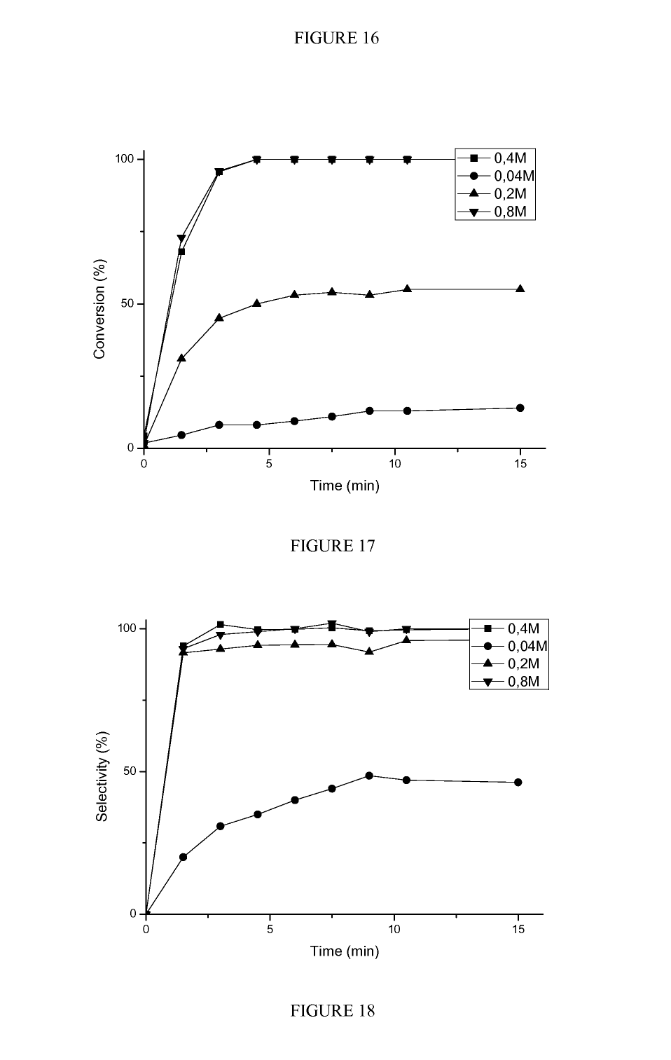

The time of the base addition has been varied. The aqueous solution of K₂CO₃ which pH was adjusted until 10.6 with HCl was used. 15 mL of this solution was added over 1.5, 2, 2.5, 3 and 3.5 min (FIGS. 15 and 16). The conversion decreased when the base was added slowly. However, the selectivity remained constant whatever the duration of addition.

e. Influence of the Base Concentration

The concentration of K₂CO₃ has been varied. The concentrations tested were 0.04, 0.2, 0.4 and 0.8 M (FIGS. 17 and 18). The results showed that the best concentration is 0.4M.

f. Influence of the Reaction

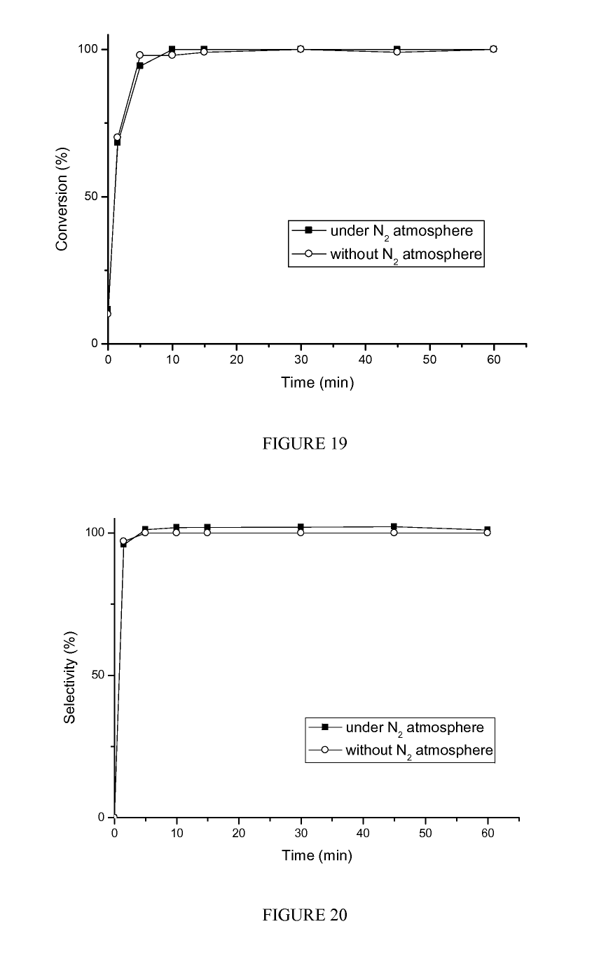

The reaction was carried out with the Pd(5%)/TiO₂ WI catalyst of Example 1, a solution of K₂CO₃ at a concentration of 0.4M and added over 1.5 min , under inert atmosphere (nitrogen) or under non-inert atmosphere (air). The conversion (FIG. 19) and selectivity (FIG. 20) results are identical in both cases. Results show that the reactions can be performed indifferently with or without an inert atmosphere.

Furthermore, the formation of toluene was not observed in any of the above listed reactions. This means that there was no dehalogenation side reaction of the aryl halide starting material.

### Example 4

### Suzuki-Miyaura's Catalytic Tests Reaction with Boronic Acid Esters

### Catalytic Reaction

The Pd(5%)/TiO₂ WI catalyst prepared in Example 1 was tested in the following Suzuki-Miyaura test reaction (coupling reaction 2). The reactants were 4-bromotoluene and phenylboronic acid pinacol ester. The desired product is 4-methylbiphenyl.

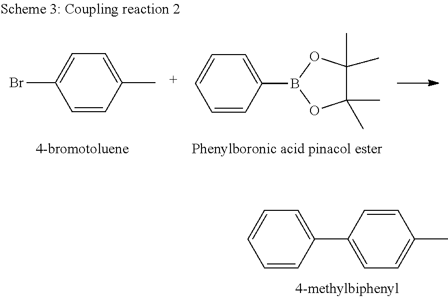

The procedure used in this catalytic test is identical to the one described in Example 3. Once the working temperature (95° C.) was reached, 15 mL of K₂CO₃ solution was added using a peristaltic pump over 1.5±0.10 min.

### Test Results

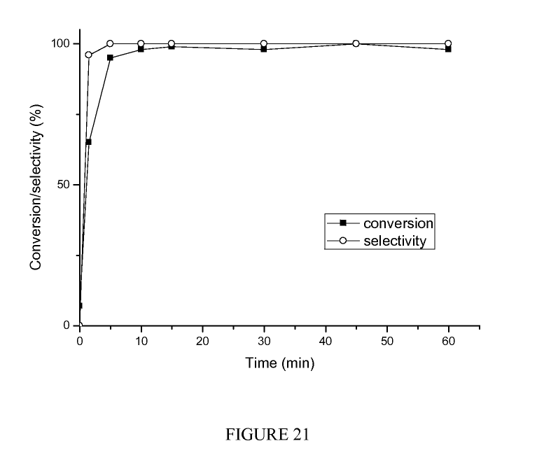

FIG. 21 shows the 4-bromotoluene conversion and the 4-methylbiphenyl selectivity obtained for coupling reaction 2. Those results indeed confirm that the method of the invention can be carried out with boronic acid esters. As with boronic acids, the formation of toluene was not observed in any of the above listed reactions. This means that there was no dehalogenation side reaction of the aryl halide starting material.

1-10. (canceled)

11. Method of carrying out Suzuki-Miyaura CC-coupling reactions, comprising reacting an aryl halide with an aryl boronic acid or en ester thereof, in an organic solvent in the presence of an oxide supported palladium catalyst and a base, characterized in that the base is added in the form of an aqueous solution with a constant flow rate within a predetermined period of time at the beginning of the reaction.

12. The method according to claim 11, wherein the base is added at a flow rate of 0.133 to 0.2 L/(min*L reactor volume).

13. The method according to claim 11, wherein the base is added within a period of time of 1 to 2 minutes.

14. The method according to claim 11, wherein the aqueous solution of the base is buffered at a pH of 8 to 12.

15. The method according to claim 11, wherein the base is an inorganic base.

16. The method according to claim 15, wherein the base is selected from the group consisting of solutions of K₂CO₃, Na₂CO₃, Cs₂CO₂, NaOH, KOH, Na₃PO₄, and KF.

17. The method according to claim 11, wherein the oxide support of the catalyst is selected from the group consisting of γ-Al₂O₂, TiO₂, ZrO₂, CeO₂, MgO, SiO₂, and SiO₂—P.

18. The method according to claim 17, characterized in that the oxide support is TiO₂.

19. The method according to claim 11, wherein the palladium is loaded on the oxide support in a quantity of 0.3 to 7% by weight with respect to the weight of the support.

20. The method according to claim 11, wherein the reaction is carried out at a temperature of between 70° C. and 100° C.

21. The method according to claim 11, wherein the base is added at a flow rate of 0.150 to 0.183 L/(min*L reactor volume).

22. The method according to claim 11, wherein the base is added at a flow rate of about 0.167 L/(min*L reactor volume).

23. The method according to claim 11, wherein the base is added within a period of time of 1.25 to 1.55 minutes.

24. The method according to claim 11, wherein the base is added within a period of time of about 1.5 minutes.

25. The method according to claim 11, wherein the aqueous solution of the base is buffered at a pH of 10 to 11, more preferably about 10.6.

26. The method according to claim 11, wherein the aqueous solution of the base is buffered at a pH of about 10.6.

27. The method according to claim 11, wherein the reaction is carried out at a temperature of between 78° C. and 98° C.

28. The method according to claim 11, wherein the reaction is carried out at a temperature of between between 80° C. and 95° C. and most preferably about 95° C.

29. The method according to claim 11, wherein the reaction is carried out at a temperature of about 95° C.
